SEQUENCE LISTING (3 sequences)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  Conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA

(19) United States
(12) Patent Application Publication
(10) Pub. No.: US 2016/0130321 A1
(43) Pub. Date: May 12, 2016

(54) USE OF A VEGF ANTAGONIST IN TREATING MACULAR EDEMA

(71) Applicant: Gabriela BURIAN, Basel (CH)

(72) Inventor: Gabriela Burian, Basel (CH)

(21) Appl. No.: 14/898,344

(22) PCT Filed: Jun. 18, 2014
(86) PCT No.: PCT/IB2014/062372

§ 371 Date: Dec. 14, 2015

Related U.S. Application Data
(60) Provisional application No. 61/837,253, filed on Jun. 20, 2013.

(51) Int. Cl. C07K 14/71; A61K 45/06; A61K 39/395; A61K 38/17

(52) U.S. Cl. CPC C07K 14/71; A61K 38/179; A61K 45/06; A61K 39/3955; C07K 2319/30

(57) ABSTRACT

The present invention relates to the use of a non-antibody VEGF antagonist in the treatment of macular edema secondary to diseases or conditions other than diabetes or retinal vein occlusion.

### TECHNICAL FIELD

This invention is in the field of treating retinal disorders. In particular, the present invention relates to the treatment of macular edema secondary to diseases or conditions other than diabetes or retinal vein occlusion.

### BACKGROUND ART

Disruption of the blood-retinal barrier (which may occur during inflammatory processes in the eye) has been shown to result in fluid accumulation within the retina both intra- and extracellularly (Yanoff et al. (1984) Sury Ophthalmol. 28 Suppl:505-11). While the exact pathogenesis is not well understood, macular edema is caused by the accumulation of excess fluid within the macular retina.

Macular edema is the most frequent complication in uveitis. Uveitic macular edema (uveitic ME) may occur secondarily to autoimmune (pars planitis), infectious (toxoplasmosis), toxic (rifabutin-associated), idiopathic (sarcoidosis) conditions. For example, diseases such as sarcoidosis, birdshot retinochoroidopathy, Behcet's syndrome, toxoplasmosis, Eales' disease, idiopathic vitritis, Vogt-Koyanagi-Harada syndrome, and scleritis have been shown to be associated with uveitic ME. Standard treatment for uveitic ME is the administration of topical or oral non-steroidal anti-inflammatory drugs (NSAIDs). However, in some cases, uveitic ME persists, and steroids as well as intravitreal injections of triamcinolone have been used to control the disease.

In patients suffering from uveitic ME, intravitreous vascular endothelial growth factor (VEGF) concentrations have been shown to be increased (Fine et al. (2001) Am J Ophthalmol. 132(5):794-6, Weiss et al. (2009) Eye (Lond). 23(9):1812-8). This finding has provided a rationale for treating uveitic ME with antibodies directed against VEGF. For example, patients with uveitic ME refractory to standard treatments with anti-inflammatory drugs have been treated with some success using intravitreal bevacizumab injections (Bae et al. (2011) Retina 31(1):111-8). Combination therapy of intravitreal bevacizumab and triamcinolone has also been reported (Cervantes-Castañeda et al. (2009) Eur J Ophthalmol. 19(4):622-9).

Macular edema is also commonly observed after cataract surgery and has also been described as Irvine-Gass syndrome. Pseudophakic and aphakic macular edema has been observed. The standard course of treatment of post-operative macular edema is anti-inflammatory therapy with topical corticosteroids or NSAIDs. Refractory cases of post-operative macular edema can be treated with intravitreal injections of triamcinolone.

Intravitreal triamcinolone therapy in uveitic ME is frequently associated with increases in intraocular pressure and cataract progression (Kok (2005) Ophthalmology 112(11):1916.e1-7). The effects of intravitreal bevacizumab injections are generally short-lived (Barkmeier & Akduman (2009) Ocul Immunol Inflamm. 17(2):109-17, Bae et al. (2011) Retina 31(1):111-8). Similarly, treatment of macular edema secondary to cataract surgery using intravitreal anti-VEGF therapy with bevacizumab frequently only results in a short-term improvement of visual acuity (Buchholz et al. (2010) Dev Ophthalmol. 46:111-22).

A need for more efficacious therapies with long-term prevention of recurring macular edema secondary to uveitis and cataract surgery exists. The present invention addresses the problem by providing alternative treatment modalities that improve the overall outcome of anti-VEGF therapy, reduce the number of VEGF antagonist injections needed and provide a long-lasting effect.

### DISCLOSURE OF THE INVENTION

The present invention relates to novel treatments of macular edema from causes other than diabetes or retinal vein occlusion. In particular, the present invention relates to the use of a non-antibody VEGF antagonist in the treatment of macular edema secondary to uveitis or cataract surgery. The invention also provides treatment schedules that yield a better overall disease outcome such as stabilization or improvement of visual acuity. The disclosed therapies delay or ideally prevent recurrence of ME.

The invention also provides a non-antibody VEGF antagonist for use in a method for treating a patient having macular edema secondary to a disease or condition other than diabetes or retinal vein occlusion, wherein said method comprises administering to the eye of a patient a non-antibody VEGF antagonist. The non-antibody VEGF antagonist may be administered intravitreally, e.g. through injection, or topically, e.g. in form of eye drops.

The invention further provides the use of a non-antibody VEGF antagonist in the manufacture of a medicament for treating a patient having macular edema secondary to a disease or condition other than diabetes or retinal vein occlusion.

Non-Antibody VEGF Antagonists

VEGF is a well-characterised signal protein which stimulates angiogenesis. Two antibody VEGF antagonists have been approved for human use, namely ranibizumab (Lucentis®) and bevacizumab (Avastin®). Treatment of uveitic ME patients with bevacizumab resulted in a transient positive effect (Weiss et al. (2009) Eye (Lond). 23(9):1812-8). Similarly, treatment with bevacizumab has been shown to at least temporarily improve post-operative pseudophakic macular edema in patients having undergone cataract surgery suggesting an involvement of VEGF in the disease process (reviewed in: Barkmeier & Akduman (2009) Ocul Immunol Inflamm 17(2):109-17).

In one aspect of the invention, the non-antibody VEGF antagonist is an immunoadhesin. One such immuoadhesin is aflibercept (Eylea®), which has recently been approved for human use and is also known as VEGF-trap (Holash et al. (2002) PNAS USA 99:11393-98; Riely & Miller (2007) Clin Cancer Res 13:4623-7s). Aflibercept is the preferred non-antibody VEGF antagonist for use with the invention. Aflibercept is a recombinant human soluble VEGF receptor fusion protein consisting of portions of human VEGF receptor extracellular domains 1 and 2 fused to the Fc portion of human IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR and domain 2 from VEGFR1/Flt-1; these domains are fused to a human IgG Fc protein fragment (Li et al., 2011 Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MVSYWDTGVLLCALLSCLLLTGSSSGGRPFVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
LPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSFFLYSKLTVDKSRWQQGNVFSCSVMHEALHNHYTQKSLSLSP
GK

and, like VEGF-trap, can be present as a dimer. This fusion protein and related molecules are further characterized in EP1767546.

Other non-antibody VEGF antagonists include antibody mimetics (e.g. Affibody® molecules, affilins, affitins, anticalins, avimers, Kunitz domain peptides, and monobodies) with VEGF antagonist activity. This includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSDLGKKLLEAARAGQDDEVRILMANGADVNTADSTGWTPLHLAVPWGHL
EIVEVLLKYGADVNAKDFQGWTPLHLAAAIGHQEIVEVLLKNGADVNAQD
KFGKTAFDISIDNGNEDLAEILQKAA

Recombinant binding proteins comprising an ankyrin repeat domain that binds VEGF-A and prevents it from binding to VEGFR-2 are described in more detail in WO2010/060748 and WO2011/135067.

Further specific antibody mimetics with VEGF antagonist activity are the 40 kD pegylated anticalin PRS-050 and the monobody angiocept (CT-322).

The non-antibody VEGF antagonist may be modified to further improve their pharmacokinetic properties or bioavailability. For example, a non-antibody VEGF antagonist may be chemically modified (e.g., pegylated) to extend its in vivo half-life. Alternatively or in addition, it may be modified by glycosylation or the addition of further glycosylation sites not present in the protein sequence of the natural protein from which the VEGF antagonist was derived.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO: 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

Non-antibody VEGF antagonists are preferred herein over antibody VEGF antagonists due their different pharmacokinetic profile when administered intravitreally. Preferably, the non-antibody VEGF antagonist of the invention binds to VEGF via one or more protein domain(s) that are not derived from the antigen-binding domain of an antibody. The non-antibody VEGF antagonist of the invention are preferably proteinaceous, but may include modifications that are non-proteinaceous (e.g., pegylation, glycosylation).

Patient

In one aspect of the invention, non-antibody VEGF antagonists are particularly useful for treating macular edema from causes other than diabetes or retinal vein occlusion. For example, uveitic ME may occur secondarily to an autoimmune disease (e.g., pars planitis), an infection (e.g., toxoplasmosis), or an adverse drug reaction (e.g., to rifabutin). In some cases, macular edema will not be associated with any disease or condition (idiopathic macular edema). Macular edema may be associated with a number of diseases including sarcoidosis, birdshot retinochoroidopathy, Behcet's syndrome, Eales' disease, idiopathic vitritis, Vogt-Koyanagi-Harada syndrome, retinitis pigmentosa, and scleritis. Macular edema may be of cystoid or non-cystoid appearance. Macular edema may also be associated with radiation retinopathy. Radiation retinopathy can cause decreased vision in patients who have received either external beam radiation or local plaque therapy to the eye. Treatment with the non-antibody VEGF antagonists of the invention may be particularly suitable for patients suffering from macular edema secondary to Behcet's uveitis. Patients suffering from macular edema after cataract surgery may also be treated with the non-antibody VEGF antagonist therapies according to the invention. Patients include those with Irvine-Gass syndrome, aphakic macular edema and pseudophakic macular edema.

A patient's medical history is usually used to determine the underlying cause for the development of macular edema. The medical history as well as previous treatments may inform specific treatment options, in particular for combination treatments. For example, for patients in whom macular edema may be triggered by an inflammatory response, combination therapy with an anti-inflammatory agent can be considered. The patient's age, family history and diagnostic testing for the above mentioned diseases can further be used to aid diagnosis of macular edema that is secondary to causes other than diabetes or retinal vein occlusion.

Patients suffering from macular edema and being at risk for an increase of intraocular pressure benefit from non-antibody VEGF antagonist treatment according to the invention. Previous treatment with triamcinolone may increase the risk of increased intraocular pressure.

In a particular aspect of the invention, patients that failed to respond to standard anti-inflammatory therapy for macular edema or have become refractory to anti-inflammatory drugs administered systemically, topically or intravitreally for the treatment of macular edema may respond well to the non-antibody VEGF antagonist therapy of the invention. Patients who previously were administered bevacizumab intravitreally and presented with recurrent macular edema after 4, 6 or 8 weeks may not require treatment for an interval extended by 2, 4 or ideally 6 weeks after administration with the non-antibody VEGF antagonist of the invention. Patients with uveitic ME who show an extensive leakage from the choroid or a leakage of the optic disk may respond well to the non-antibody VEGF antagonist. For this group of patients, combination therapy with triamcinolone may be particularly advantageous.

The therapies of the present invention are particularly well suited for patients with macular edmea who are younger than 65 years, preferably younger than 55 years. Generally, the earlier treatment is started, the better the outcome of the therapy will be when using the non-antibody VEGF antagonist of the invention.

Administration

The non-antibody VEGF antagonist of the invention will generally be administered to the patient via intravitreal injection, though other routes of administration may be used, such as a slow-release depot, an ocular plug/reservoir or eye drops. Administration in aqueous form is usual, with a typical volume of 20-150 μl e.g. 40-60 μl, or 50 μl. Injection can be via a 30-gauge×½-inch (12.7 mm) needle. For example, aflibercept is generally administered via intravitreal injection at a dose of 2 mg (suspended in 0.05 mL buffer comprising 40 mg/mL in 10 mM sodium phosphate, 40 mM sodium chloride, 0.03% polysorbate 20, and 5% sucrose, pH 6.2). However, the normal dose may be reduced for the treatment of smaller children and in particular infants. The dose for treating an infant with a VEGF antagonist of the invention is usually 50% of the dose administered to an adult. Smaller doses (e.g., 0.5 mg per monthly injection) may also be used.

Alternatively, an intravitreal device is used to continuously deliver a non-antibody VEGF antagonist into the eye over a period of several months before needing to be refilled by injection. Various intravitreal delivery systems are known in the art. These delivery systems may be active or passive. For example, WO2010/088548 describes a delivery system having a rigid body using passive diffusion to deliver a therapeutic agent. WO2002/100318 discloses a delivery system having a flexible body that allows active administration via a pressure differential. Alternatively, active delivery can be achieved by implantable miniature pumps. An example for an intravitreal delivery system using a miniature pump to deliver a therapeutic agent is the Ophthalmic MicroPump System™ marketed by Replenish, Inc. which can be programmed to deliver a set amount of a therapeutic agent for a pre-determined number of times.

The non-antibody VEGF antagonist is typically encased in a small capsule-like container (e.g., a silicone elastomer cup). The container is usually implanted in the eye above the iris. The container comprises a release opening. Release of the non-antibody VEGF antagonist may be controlled by a membrane positioned between the non-antibody VEGF antagonist and the opening, or by means of a miniature pump connected to the container. Alternatively, the non-antibody VEGF antagonist may be deposited in a slow-release matrix that prevents rapid diffusion of the antagonist out of the container.

Preferably, the intravitreal device is designed to release the non-antibody VEGF antagonist at an initial rate that is higher in the first month. The release rate slowly decreases, e.g., over the course of the first month after implantation, to a rate that is about 50% less than the initial rate. The container may have a size that is sufficient to hold a supply of the non-antibody VEGF antagonist that lasts for about four to six months. Since a reduced dose of VEGF antagonist may be sufficient for effective treatment when administration is continuous, the supply in the container may last for one year or longer, preferably about two years, more preferably about three years.

Continuous administration via an intravitreal device may be particularly suitable for patients with chronic macular edema secondary to uveitis or macular edema refractory to conventional treatment with anti-inflammatory therapy. Because only a small surgery is required to implant a delivery system and intravitreal injections are avoided, patient compliance issues with repeated intravitreal injections can be avoided. Intravitreal concentrations of the non-antibody VEGF antagonist are reduced, and therefore the potential risk of side-effects from non-antibody VEGF antagonist entering the circulation is decreased. This aspect may be of a particular advantage in children who may require general anaesthesia for intravitreal injections. Systemically elevated non-antibody VEGF antagonist levels may interfere with normal growth and development of children who therefore may benefit from lower intravitreal concentrations of the non-antibody VEGF antagonist.

In one aspect of the invention, the non-antibody VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile antagonist solution prior to administration can be avoided. Pre-filled syringes also provide easier handling for the administering ophthalmologist.

Slow-Release Formulations

Non-antibody VEGF antagonist may be provided as slow-release formulations. Slow-release formulations are typically obtained by mixing a therapeutic agent with a biodegradable polymer or encapsulating it into microparticles. By varying the manufacture conditions of polymer-based delivery compositions, the release kinetic properties of the resulting compositions can be modulated.

A slow-release formulation in accordance with the invention typically comprises a non-antibody VEGF antagonist, a polymeric carrier, and a release modifier for modifying a release rate of the non-antibody VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lactide-co-glycolide (PLGA), polyesters, poly (orthoester), poly(phosphazine), poly (phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

Preferably, the non-antibody VEGF antagonist is incorporated into a microsphere-based sustained release composition. The microspheres are preferably prepared from PLGA. The amount of non-antibody VEGF antagonist incorporated in the microspheres and the release rate of the non-antibody VEGF antagonist can be controlled by varying the conditions used for preparing the microspheres.

Processes for producing such slow-release formulations are described in US 2005/0281861 and US 2008/0107694.

Treatment Regimens

Non-antibody VEGF antagonists of the invention allow increased spacing between administrations resulting in a more cost-effective therapy. In addition, better patient compliance is achieved when intravitreal injections have to be performed less frequently. This is particularly advantageous in patients suffering from chronic macular edema secondary to uveitis or cataract surgery who may require multiple injections to improve visual acuity or prevent vision loss. Patients with current macular edema after treatment with an antibody VEGF antagonist may also benefit from the treatment of the invention because recurrence of macular edema is delayed or prevented using the non-antibody VEGF antagonists of the invention.

In some cases, a single injection of the non-antibody VEGF antagonist according to the invention may be sufficient to ameliorate the disease or prevent disease progression for many years. In other cases, three injections each one month apart are administered to the patient, while any subsequent injections are performed less frequently or on an as needed-basis. In certain cases, two injections spaced 6 weeks apart, preferably 8 weeks apart, more preferably 10 weeks apart may be required to improve visual acuity or halt disease progression. In other cases, three or more injections may be needed. In these cases, the time between injections should be at least 6 weeks, preferably 8 weeks, more preferably 10 weeks apart.

Disease progression or recurrence of macular edema may require one or more or continued treatment cycles. For example, in a first cycle, two or more injections spaced 4 weeks, 6 weeks, preferably 8 weeks, more preferably 10 weeks apart may be administered followed by an interruption of treatment for 3 months, 4 months, 5 months, 6 months, 9 months, 12 months, 24 months or 36 months. If macular edema reappears, the treatment is continued with a second cycle. In one embodiment, the treatment may comprise two or more (preferably 3) injections spaced 4 weeks apart, followed by two or more injections spaced 8 weeks apart.

In another aspect of the invention, the non-antibody VEGF antagonist according to the invention is administered as needed. The non-antibody VEGF antagonist is administered the first time after an initial diagnosis of macular edema has been made. A diagnosis of macular edema can be made during examination of the eye by a combination of slit-lamp evaluation and biomicroscopic fundus examination with optical coherence tomography (OCT) and/or fluorescein fundus angiography. A second, third or further administration of the non-antibody VEGF antagonist is performed only if examination of the eye reveals signs of persistent or recurring macular edema.

Combination Therapy

In an alternative aspect of the invention, treatment time and patient compliance is improved by using a non-antibody VEGF antagonist in combination with an anti-inflammatory agent. Administering the VEGF antagonist in combination with an anti-inflammatory agent can have synergistic effects depending on the underlying cause of the macular edema. Addition of an anti-inflammatory agent is particularly advantageous in macular edema secondary to an inflammatory disease or condition. Anti-inflammatory agents include steroids and NSAIDs. NSAIDs used in the treatment of ocular diseases include ketorolac, nepafenac and diclofenac. In some instances, the use of diclofenac is preferred. Corticosteroids used in treating ocular diseases include dexamethasone, prednisolone, fluorometholone and fluocinolone. Other steroids or derivatives thereof that may be used in combination with VEGF antagonist treatment include anecortave, which has angiostatic effects but acts by a different mechanism than the VEGF antagonists according to the invention. A preferred anti-inflammatory agent is triamcinolone. The anti-inflammatory agent may also be a TNF-α antagonist. For example, a TNF-α antibody may be administered in combination with a non-antibody VEGF antagonist. TNF-α antibodies, e.g. those sold under the trade names Humira®, Remicade®, Simponi® and Cimzia®, are well known in the art. Alternatively, a TNF-α non-antibody antagonist such as Enbrel® may be administered in combination with a non-antibody VEGF antagonist of the invention.

The anti-inflammatory agent may be administered at the same time as the non-antibody VEGF antagonist. The anti-inflammatory agent can be administered systemically or locally. For example, the anti-inflammatory agent may be administered orally, topically, or, preferably, intravitreally. In a preferred embodiment, triamcinolone is administered intravitreally at the same time as the non-antibody VEGF antagonist.

In yet another aspect of the invention, the non-antibody VEGF antagonist is administered after administration of an antimicrobial agent. For example, the antimicrobial agent may be selected from gatifloxacin, ciprofloxacin, ofloxacin, norfloxacin, polymixin B+chloramphenicol, chloramphenicol, gentamicin, fluconazole, sulfacetamide, tobramycin, neomycin+polymixin B, and netilmicin. Alternatively, the antimicrobial agent may be selected from pyrimethamine, sulfadiazine and folinic acid or a combination thereof. Combination with pyrimethamine can be particularly advantageous in treating patient with macular edema associated with toxoplasmosis.

General

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

### MODES FOR CARRYING OUT THE INVENTION

### Comparative Example

Intravitreal administration of ranibizumab was tested in 7 patients with controlled uveitis and refractory cystoid macular edema who had previously failed both oral and regional corticosteroid treatment. Intravitreal ranibizumab injections (0.5 mg) were given monthly for the first 3 months. Subsequently, treatment was continued as needed. The mean change in best-corrected visual acuity (BCVA) from baseline was determined at 3 months and re-evaluated after 6 months of treatment. In addition, the mean change in central retinal thickness (CRT) was determined at both 3 months and 6-months using ocular coherence tomography (OCT).

At 3 months, the mean increase in acuity for the 6 patients who completed follow-up was 13 letters (2.5 lines). The mean decrease in CRT was 357 μm. Both BCVA and CRT improved significantly between baseline and 3 months (P=0.03 for each). Most patients required reinjection after the 3-month initial treatment. Nevertheless, the improvement in BCVA and CRT was maintained at 6 months. No significant ocular or systemic adverse effects were observed during the study.

### Example 1

This study evaluates aflibercept for the treatment of macular edema associated with retinopathy secondary to previous radiation treatment.

Male and female patients, 18 years and older, diagnosed with macular edema associated with retinopathy secondary to previous radiation therapy were studied. Patients are suitable for inclusion who have (i) retinopathy associated with previous 1-125 brachytherapy for uveal melanoma (typically 85 Gy over 96 hours) at least 6 months prior to enrolment; (ii) center involved macular edema>300 μm in thickness on SD-OCT; (iii) best corrected visual acuity of 20/40-20/400; (iv) for females of child-bearing age: undergo birth control therapy.

Patients are excluded who (i) demonstrated pre-existing retinopathy due to other disorders; (ii) have vision decrease, which is considered to be due to ischemic radiation retinopathy without macular edema or optic neuropathy; (iii) are detected a presence of metastasis; (iv) are, in the case of females, pregnant (detected by positive pregnancy test) or in lactation period; (v) are premenopausal women which are not using adequate contraception (the following are considered effective means of contraception: surgical sterilization or use of oral contraceptives, barrier contraception with either a condom or diaphragm in conjunction with spermicidal gel, an IUD, or contraceptive hormone implant or patch); (vi) did prior enrollment in any study with intravitreal aflibercept injection; (viii) any other condition that the investigator believes would pose a significant hazard to the subject if the investigational therapy were initiated; (ix) demonstrate uncontrolled glaucoma in the study eye (defined as IOP≧30 mmHg despite treatment with anti-glaucoma medication; (x) have a history of cerebral vascular accident, myocardial infarction, transient ischemic attacks within 4 months of study enrolment; (xi) have active infectious conjunctivitis, keratitis, scleritis, or endophthalmitis in either eye; (xii) have any concurrent intraocular condition in the study eye (e.g., cataract or diabetic retinopathy) that, in the opinion of the investigator, could either require medical or surgical intervention during the study period to prevent or treat visual loss that might result from that condition, or if allowed to progress untreated, could likely contribute to loss of at least 2 Snellen equivalent lines of BCVA over the 6-month study period; (xiii) demonstrated presence of significant subfoveal fibrosis or atrophy; (xiv) had intraocular surgery (including cataract surgery) in the study eye within 2 months of enrolment; (xv) have active intraocular inflammation (grade trace or above) in the study eye; (xvi) have a history of allergy to fluorescein, ICG or iodine, not amenable to treatment; (xvii) have undergone prior/Concomitant Treatment; (xviii) have undergone panretinal photocoagulation treatment; (xix) have received previous intraocular steroids or PDT within 3 months; (xx) previously participated in any studies of investigational drugs within 30 days preceding Day 0 (excluding vitamins and minerals); (xxi) have undergone previous treatment with intravitreally (in either eye) or intravenously administered Avastin (bevacizumab) within 60 days or concomitant use in either eye outside the scope of this study; (xxii) have received Eylea, Macugen or Lucentis treatment in study eye within previous 60 days or concomitant use in either eye outside the scope of this study; (xxiii) have undergone prior submacular or vitreous surgery.

Patients are randomized to two treatment groups: (1) intravitreal injection of aflibercept 2 mg dosed monthly; (2) intravitreal injection of aflibercept 2 mg dosed every two months (after 3 initial monthly injections). Patients in both groups are followed up until month 12.

The primary outcome is the rate of incidents and severity of adverse events within a 12 month time frame. Secondary outcome measures are (i) mean change in best corrected visual acuity (BCVA) from baseline; (ii) mean change in central foveal thickness by optical coherence tomography (OCT) from baseline; (iii) mean visual acuity; (iv) mean central foveal thickness; (v) proportion of patients gaining 3 lines of vision; (vi) mean change in lesion characteristics (lesion size, leakage); (vii) proportion of patients with no fluid on OCT; (viii) mean change in macular volume.

Interim Results

Of three patients currently undergoing treatment in the study, one patient had a Grade I adverse reaction of nausea/syncope during an injection. The other two patients had no adverse events. Visual acuity improved in all subjects, from 20/100 to 20/50, 20/125 to 20/40, and 20/30 to 20/20, respectively. Mean central foveal thickness and macular volume by OCT were both improved in all subjects.

It will be understood that the invention is described above by way of example only and modifications may be made whilst remaining within the scope and spirit of the invention.

1. A method for treating a patient having macular edema secondary to a disease or condition other than diabetes or retinal vein occlusion, comprising administering to the patient a therapeutically effective amount of a non-antibody VEGF antagonist.

2. The method of claim 1, wherein the macular edema is secondary to an inflammatory condition.

3. The method of claim 1, wherein the macular edema is cystoid macular edema (ME).

4. The method of claim 2, wherein the inflammatory condition is triggered by an infectious agent or an autoimmune response.

5. The method of claim 1, wherein the macular edema is secondary to pars planitis, sarcoidosis, birdshot retinochoroidopathy, Behcet's syndrome, toxoplasmosis, Eales' disease, idiopathic vitritis, Vogt-Koyanagi-Harada syndrome, and scleritis.

6. The method of claim 1, wherein the macular edema is associated with retinopathy.

7. The method of claim 6, wherein the retinopathy is secondary to previous radiation treatment.

8. The method of claim 6, wherein the retinopathy is associated with previous brachytherapy for uveal melanoma.

9. The method of claim 1, wherein the non-antibody antagonist is selected from a recombinant human soluble VEGF receptor fusion protein and a recombinant binding protein comprising an ankyrin repeat domain that binds VEGF-A.

10. The method of claim 1, wherein the non-antibody VEGF antagonist is aflibercept.

11. The method of claim 10, wherein aflibercept is administered via intravitrial injection.

12. The method of claim 10, wherein the aflibercept is administered at a dose of 2 mg.

13. The method of claim 1, wherein the method further comprises administering an anti-inflammatory agent.

14. The method of claim 13, wherein both the non-antibody VEGF antagonist and the anti-inflammatory compound are administered intravitreally.

15. The method of claim 13, wherein the anti-inflammatory agent is administered at the same time as the non-antibody VEGF antagonist.

16. The method of claim 1, wherein the non-antibody VEGF antagonist is administered every 4 weeks, every 6 weeks, every 8 weeks or every 10 weeks.

17. The method of claim 16, wherein the non-antibody VEGF antagonist is administered every 4 weeks.

18. The method of claim 1, wherein the non-antibody VEGF antagonist is administered two or more times, preferably three times, every 4 weeks, followed by two or more administrations every 8 weeks.

19. The method of claim 1, wherein the non-antibody VEGF antagonist is administered continuously.

20. The method of claim 1, wherein a first dose of the non-antibody VEGF antagonist is administered after the initial diagnosis of macular edema and wherein a second dose of the non-antibody VEGF antagonist is administered only if the macular edema persists or recurs after administration of the first dose.

21. The method of claim 20, wherein the interval between the first and the second treatment is at least 4 weeks, at least 6 weeks, at least 8 weeks or at least 10 weeks.

22. The method of claim 20, wherein the interval between the first and the second treatment is at least 3 months, 6 month or 9 months.
